(19) United States
(12) Patent Application Publication
(10) Pub. No.: US 2009/0048667 A1
(43) Pub. Date: Feb. 19, 2009

(54) Controlled Drug-Release Composition and Drug-Releasable Medical Device

(75) Inventors: Akira Mochizuki, Shizuoka (JP); Shuzou Yamashita, Okayama (JP)

Correspondence Address: THE WEBB LAW FIRM, P.C., 700 KOPPERS BUILDING, 436 SEVENTH AVENUE, PITTSBURGH, PA, 15219, US

(73) Assignees: TOKAI UNIVERSITY EDUCATIONAL SYSTEM, Tokyo (JP); JAPAN STENT TECHNOLOGY CO., LTD., Okayama-Shi (JP)

(21) Appl. No.: 12/093,889

(22) PCT Filed: Nov. 15, 2006
(86) PCT No.: PCT/JP2006/322718

§ 371 Date: May 15, 2008

(30) Foreign Application Priority Data
Nov. 16, 2005 (JP) 2005-331503
May 9, 2006 (JP) 2006-130786

(51) Int. Cl. A61L 33/06; A61K 47/32; A61F 2/82; A61M 25/00; A61B 17/03; A61B 5/00

(52) U.S. Cl. 623/1.43; 514/772.3; 514/772.6; 604/523; 606/151; 600/300

(57) ABSTRACT

A drug-releasable medical device contains a controlled drug-release composition comprising 100 parts by weight of an organic polymeric material which is soluble in an organic solvent and insoluble in water, 5 to 60 parts by weight of a lipid-soluble, low molecular weight release auxiliary agent and 1 to 70 parts by weight of a drug. When the composition is applied on a stent, a catheter, an organ replacement medical device, an artificial organ or the like in the form of coating or the like, the medical device is provided with a drug release function. Argatroban or sarpogrelate hydrochloride or both of them are gradually released from the surface of a stent for treating coronary artery stenosis, for example. In order to exhibit a sustained-release function for a desired period of time, the drug to be gradually released is carried in a polymeric material coated on a surface of a metal forming the stent or in a porous stent substrate.

### TECHNICAL FIELD

The present invention relates to a controlled drug-release composition and the like and, in particular, to a controlled drug-release composition which imparts a drug release function to a medical device and the like and a drug-releasable medical device carrying the composition, especially a stent.

### BACKGROUND ART

Recently, the technology development of a drug formulation and administration has progressed so that not only new drugs but also existing drugs can effectively exhibit efficacy. For example, there has been developed a formulation technology for releasing an active pharmaceutical ingredient after elapse of a fixed period of time by coating a drug with a special film. Based on the concept of a drug delivery system (DDS), there has been actively studied a drug formulation using a nanosphere and a microcapsule including a liposome. The function which such DDS aims at includes a controlled release property, a target directivity, easiness of ingestion and administration, effectiveness enhancement and side effect reduction, or the like.

As a drug continuously releasable material in consideration of DDS, there have been widely studied a polymer matrix material such as a poly(lactic acid), a lactic acid-glycolic acid copolymer and the like (Patent Documents 1 and 2 and Non-patent Document 1). However, it is customary that a drug release rate as desired was not obtained at the delivered site only by mixing a drug with these biodegradable polymers. The reason is that the diffusion migration rate of a drug is significantly low in such polymer matrix or a drug is difficult to liberate from there (Non-patent Document 1). To solve these problems, there has been studied a technique for securing or increasing the release amount of a drug by converting a polymer matrix to a porous body or fine particles to increase the contact area, which is into the practical use. For the above polymer matrix, the pore size control in converting to a porous body is extremely important and the sophisticated condition settings are required, therefore, the manufacturing cost is inevitably increased.

On the other hand, with the advancement of medical engineering technology, there has been studied a technique for achieving a desired purpose by incorporating, embedding or indwelling some medical tools, devices and equipments inside or outside of a living body mainly for the sake of diagnosis and treatment. It has been a rather negatively considered concept that the above-mentioned polymer matrix technique is applied directly to medical tools, for example, a catheter, a stent, an artificial blood vessel and the like. The reason is that it is difficult to form a porous structure on the surface of these medical tools by a coating technique and the concept for medical tools is in a field requiring a smooth flat surface in consideration of the reaction based on foreign substance recognition by a living body.

A stent, which is one of the medical devices applied to a living body, is used for the treatment of coronary artery occlusion and the like. That is, a stent indwelled in the blood vessel revises and supplements the incised portion as well as prevents shrinkage of the blood vessel, thereby effectively reducing the incidence of restenosis of artery occlusion patients.

Up to now, there have been made various proposals concerning the material, shape and application technique of a stent for the purpose of treatment of arterial vessel occlusion including coronary artery occlusion. Since conventional materials cannot still completely avoid the risk of restenosis and reocclusion, this is a bottleneck for the application of angioplasty using a stent. Therefore, a stent having less possibility of restenosis and reocclusion has been desired in the medical site.

In addition, there has been studied a drug-releasable type stent in which various polymeric materials are combined with an anticancer drug, an immune-suppressing drug, an antibiotic, an anticoagulant drug or the like (Non-Patent Document 2). However, as a practical problem, in such a drug-releasable type stent, it is not easy to adjust the timing of a drug to be released and the release rate, and the amount and the period to be released as desired. For example, bursty release occurs at the early stage after a stent is indwelled and a continuous and sustained release of a drug may not be achieved, or because of a problem due to the system for carrying a drug, the drug may be dropped out from the stent indwelled in a living body.

As the above-mentioned anticoagulant drug, for example, there is known argatroban as an antithrombin drug and sarpogrelate hydrochloride as an antiplatelet drug. As a medical device provided with antithrombogenicity by gradually releasing argatroban, a catheter is disclosed in Patent Document 3 and Patent Document 4. Until now, there has been no information that argatroban or sarpogrelate hydrochloride, which is a synthetic anticoagulant drug, is applied to a stent and the effect is specifically validated, and it is the situation that the required release rate of the drug such that a stent exhibits anticoagulation property is not at all known.

[Patent Document 1] Japanese Patent Laid-Open Publication No. H09 (1997)-151136
[Patent Document 2] Japanese Patent Laid-Open Publication No. H09 (1997)-255590
[Patent Document 3] Japanese Patent Laid-Open Publication No. H06 (1994)-292711
[Patent Document 4] Japanese Patent Laid-Open Publication No. H06 (1994)-292718

### [Non-Patent Document 1] “Polymer Processing” Vol. 45, No. 5, pp 222; No. 6, pp 270, 1996

### [Non-Patent Document 2] “Drug-Eluting Stent”, Igaku-Shoin, 2003

### DISCLOSURE OF THE INVENTION

### Problems to be Solved by the Invention

In consideration of the above-mentioned conditions and problems, the present inventors have earnestly studied to reach the present invention. That is, the present inventors have found that the release of a drug is accelerated by adding some kind of lipid-soluble compounds having a low molecular weight and thereby have completed the present invention. The present invention is to provide a composition which can accelerate the release of a drug and stably continues to release the drug for a long period of time, and a drug-releasable medical device to which the composition is applied.

In addition, the present inventors have earnestly studied whether a manner of carrying such a drug significantly exerts an influence on the release rate and the continuous release period. As a result, the present inventors have found that the compatibility of a drug and a polymer becomes an important point in a polymeric material carrying the drug in order to release the drug for a fixed period of time and that it is more desirable that a drug is carried on an amorphous polymer, and have completed the present invention.

Furthermore, the present inventors have found that a drug may be gradually released for a fixed period of time by converting a stent substrate to a porous body so as to retain a polymeric material carrying a drug in the pore.

An object of the present invention is to provide a drug sustained-releasable stent in which a polymeric material containing an anticoagulant drug is coated on a stent or is carried on a porous stent substrate and the drug may be continuously and gradually released for a fixed period of time.

### Means to Solve the Problems

The controlled drug-release composition of the present invention is characterized by comprising 100 parts by weight of an organic polymeric material which is soluble in an organic solvent and insoluble in water; 5 to 60 parts by weight of a lipid-soluble, low molecular weight release auxiliary agent; and 1 to 70 parts by weight of a drug.

The above-mentioned organic polymeric material preferably is biodegradable or biocompatible, or both. The organic polymeric material that is biodegradable is preferably an aliphatic polyester or an aliphatic poly(carbonate), and specifically includes poly(lactic acid), a lactic acid-glycolic acid copolymer, poly(caprolactone), poly(hydroxybutyric acid) and the like.

The above-mentioned release auxiliary agent is a carboxylic acid ester, a monoester or diester of glycerin, and preferably is an ester of an organic acid selected from citric acid, tartaric acid, malic acid or the like, or monoacetate ester or diacetate ester of glycerin.

The above-mentioned drug is a pharmaceutical product and preferably is an anticoagulant drug, an anticancer drug or an immune-suppressing agent.

The above-mentioned composition may further comprise a cell adhesion material or an endothelialization promoting agent on a surface of a medical device.

The drug-releasable medical device of the present invention is characterized by containing the above-mentioned composition.

The drug-releasable medical device preferably forms a layer of the composition on the surface.

The above-mentioned medical device preferably contacts with a living body or is incorporated or indwelled in a living body, and specifically includes a stent, a catheter, a clip, an organ replacement medical device, a capsule sensor or an artificial organ.

The stent of the present invention is characterized in that it is used for treating coronary artery stenosis and an argatroban (an antithrombin drug) or a sarpogrelate hydrochloride (an antiplatelet drug) or both of them are gradually released from the surface.

The release rate of both the above-mentioned argatroban and sarpogrelate hydrochloride preferably is 1×10⁻³ μg/mm²-h to 1 μg/mm²-h on 21 days after indwelling the stent.

In addition, the stent of the present invention is characterized in that the drug to be gradually released is carried in a polymeric material coated on the surface of a metal forming the stent or in a porous stent substrate.

The polymeric material coated on the surface of the stent is preferably amorphous.

The polymeric material coated on the surface of the stent is preferably an amorphous biodegradable polymeric material.

The above-mentioned preferred polymeric material is a poly(lactic acid) or a lactic acid-glycolic acid copolymer, which is biodegradable.

The above-mentioned polymeric material preferably further comprises a release auxiliary agent that promotes the release of a drug to be carried.

The above-mentioned auxiliary agent that promotes the release of a drug is preferably a tartrate ester or a malate ester, or a monoester or diester of glycerin.

The surface of the metal forming the stent may be a porous body and the above-mentioned drug to be gradually released may be carried in the porous body. The porous body preferably has a pore size of 0.01 nm to 300 nm in diameter.

### EFFECT OF THE INVENTION

Since the controlled drug-release composition of the present invention contains a lipid-soluble, low molecular weight release auxiliary agent, the release of the contained drug is promoted in a body. A medical device containing the composition is a drug-releasable medical device which may adjust the release timing and the release rate, and the release amount and period, and when the medical device is delivered or implanted to a predetermined body site or body surface site, the drug is then released. A drug and a medical device to be applied are not particularly limited. Therefore, the controlled drug-release composition of the present invention may impart a drug release function to various medical devices.

Since an amorphous polymeric material carrying argatroban and sarpogrelate hydrochloride is well compatible with these synthetic anticoagulant drugs, a drug releasable type stent of the present invention is unlikely to cause bursty release of the drug from the stent indwelled in a blood vessel, thereby and thus the drug is continuously released at a desired release rate.

Since the release rate of an anticoagulant drug is increased by the addition of a release auxiliary agent that promotes the release of an anticoagulant drug, a sufficient amount of the drug is released to exhibit pharmacological effects from the initial time of indwelling of a stent. Therefore, the drug releasable type stent of the present invention may effectively prevent restenosis and reocclusion of artery by both actions of a stent structure and an anticoagulant drug.

In addition, the drug is released at a desired release rate and continuously by controlling the pore diameter of the porous stent substrate.

### DETAILED EXPLANATION OF THE PRESENT INVENTION

In the present specification, a “medical device” includes a “medical tool” and means a device used in the medical field in the broadest sense of the word.

“A sustained-release property” is a property of gradually releasing an active pharmaceutical ingredient in pharmaceutical technology and is intended to prevent a drug from an initial burst in pharmaceutical design and to sustain pharmacological effects for a long period of time. In addition, “biodegradability” means a property of being relatively rapidly catabolized in a living body to decompose and disappear. Further, “biocompatibility” means a tendency of bioinertness, particularly a property having an affinity to a living body and a tendency to unlikely cause an elimination reaction resulted from recognition by the living body as a foreign substance.

By “carrying” is referred that a drug is molecularly dispersed, or is to be in an aggregation cluster having sizes from nanometer order to submicron order dispersed in a polymeric matrix or porous body. In addition, an anticoagulant drug in the present specification is also referred to as an anticoagulating agent or an anticoagulant.

Controlled Drug-Release Composition

The controlled drug-release composition of the present invention is characterized by comprising 100 parts by weight of an organic polymeric material which is soluble in an organic solvent and insoluble in water, 5 to 60 parts by weight of a lipid-soluble, low molecular weight release auxiliary agent and 1 to 70 parts by weight of a drug.

Here, “Controlled drug release” means adjustment of the timing and the rate when a drug is released at a predeterminded site in the body, and the amount and duration released or the like and has not always a sustained-release property.

Hereinafter, each ingredient contained in the present composition is explained.

—Organic Polymeric Material

As a carrier which is indwelled at a predetermined site in a living body and carries a drug to be delivered to a target site, there is used an organic polymeric material which is soluble in an organic solvent and insoluble in water.

Considering that said material is used in the inside or outside of a living body as described later, such an organic polymeric material is preferably biodegradable or biocompatible or both from the viewpoint of biological safety.

As the polymeric material meeting these requirements, a polymer which especially has no bioactivity and is biodegradable is especially preferred. The biodegradable polymer is exemplified by a hydroxycarboxylic acid homopolymer, a hydroxycarboxylic acid copolymer or a mixture thereof. As specific examples of a poly(hydroxycarboxylic acid) and the hydroxycarboxylic acid copolymer, there may be mentioned poly(lactic acid), poly(glycolic acid), lactic acid-glycolic acid copolymer, polylactide, poly(lactide-glycolide), poly(ethyleneglycol-lactide), poly(glycolic acid-caprolactone), lactic acid-ethylene glycol copolymer, poly(caprolactone), poly(lactide-caprolactone), poly(hydroxyl butylate), poly(hydroxy isobutylate), poly(valerolactone), poly(γ-hydroxyvaleric acid), poly(hydroxybutyrate-hydroxyvalerate), poly(isobutylcyanoacrylate), poly(alkylcyanoacrylate), poly(ethylenesuccinate) and the like. In addition, there may be mentioned chitin, chitosan, gelatin, cellulose-acetate-terephthalate and the like.

Among these, as a more preferable polymer as a material of the present invention, there may be mentioned an aliphatic polyester (for example, a poly(hydroxy fatty acid ester)), an aliphatic poly(carbonate) (for example, a poly(alkylene carbonate)) or a poly(caprolactone) or the like. More specifically, there may be mentioned a lactic acid-glycolic acid copolymer, a poly(lactic acid), a poly(glycolic acid), a poly(malic acid) and a copolymer thereof, a lactic acid-caprolactone copolymer and a poly(hydroxybutyric acid). These polymers may be a homopolymer or a copolymer, or a mixture thereof or a salt thereof. The biocompatible high molecular weight polymer or biodegradable high molecular weight polymer used in the present invention may be easily available or may be easily synthesized by a common synthetic method.

The above-mentioned aliphatic polyester and aliphatic polycarbonate as well as the above-mentioned poly(lactic acid) are polymers which are soluble in an aromatic-based organic solvent (benzene, toluene, xylene and the like) or a halogen-based organic solvent (methylene chloride, chloroform, carbon tetrachloride, 1,1,2-trichloroethane and the like) and are insoluble in water. In the case where a drug is dissolved in these solvents, it may be used as is. Actually, many drugs are lipid-soluble and soluble in an organic solvent. On the contrary, in the case of using a drug forming a salt, for example, sarpogrelate hydrochloride, Futhan, argatroban and the like, it is not dissolved in the above-mentioned organic solvents. For this reason, as an alternative solvent, there may be used an organic solvent such as a fluorine-based alcohol and the like. Examples of the fluorine-based alcohol include hexafluoroisopropanol, trifluoroethanol and the like.

—Release Auxiliary Agent

In a drug delivery system, it is known that the gradual release rate of certain kinds of drugs is increased by adding tributyl citrate, glycerin or a long-chain fatty acid ester to a polymeric material which is a base (addition of tributyl citrate and glycerin; Journal of Biomedical Materials Research, vol. 13, pp 497-507 (1979), addition of a long-chain fatty acid ester: Journal of Controlled Release vol. 58, pp 133-141, (1999)).

A drug release rate as desired may not be obtained at the delivered site only by mixing a drug with a polymer such as a poly(lactic acid), a lactic acid-glycolic acid copolymer which are the above-mentioned organic polymeric materials. The present invention is based on the findings that the release of a drug is accelerated from a solidified composition formed by volatilization of a solvent by adding some kind of lipid-soluble, low-molecular-weight compounds. In the composition of the present invention, an auxiliary agent promoting such drug release is added together with an organic polymeric material as a carrier and a drug, thereby exhibiting the effects. In other words, in the controlled drug-release composition of the present invention, a drug is not simply sustained-released, but also the timing and the rate when the drug is released at a predetermined site in the body, and the amount and duration released may be adjustable.

A lipid-soluble, low molecular weight release auxiliary agent used for the controlled drug-release composition may be selected from the viewpoint of the drug release effect and safety. As for the safety of the auxiliary agent, a substance which itself has low biological toxicity and is almost metabolized in a living body or is excreted outside of a living body without being accumulated at all and metabolized is preferred. As the compound meeting these requirements, there may be mentioned an aliphatic carboxylic acid ester or an ester compound having a hydroxyl group in the molecule. For example, an aliphatic carboxylic acid ester having a hydroxyl group in the molecule or a polyhydric alcohol-based ester such as glycerin and the like is preferred. Specifically, the ester includes a carboxylic acid ester of acetic acid, propionic acid and the like having 2 to 6 carbon atoms and especially an ester of an organic acid selected from citric acid, tartaric acid or malic acid, or a diester and a monoester of a polyhydric alcohol such as glycerin and the like is preferred.

The chain length of the alkyl group of these esters is 1 to 12 carbon atoms long and preferably 1 to 6 carbon atoms long. Among these, methyl group, ethyl group, propyl group, butyl group and the like are preferred from the viewpoint of the easiness of availability and the compatibility with a drug and the above-mentioned organic polymeric materials.

The suitable release auxiliary agent preferably includes, for example, tartaric acid diesters or tartaric acid halfesters such as dimethyl tartrate, diethyl tartrate, dipropyl tartrate, monomethyl tartrate, monoethyl tartrate, monopropyl tartrate and the like; malic acid monoesters or malic acid diesters such as dimethyl malate, diethyl malate, dipropyl malate, monomethyl malate, monoethyl malate, monopropylmalate and the like; citric acid diesters or citric acid monoesters such as dimethyl citrate, diethyl citrate, dipropyl citrate, monomethyl citrate, monoethyl citrate, monopropyl citrate, monobutyl citrate and the like; a partial acetate ester of glycerin (for example, monoacetin, diacetin and the like); or the like.

The additive amount of the lipid-soluble, release auxiliary agent having a low molecular weight is 5 to 60 parts by weight and preferably 10 to 40 parts by weight, based on 100 parts by weight of the above-mentioned organic polymeric material. When the amount is within the above range, the release rate and the like of a drug may be controlled while maintaining the properties of the composition and the mechanical strength of the polymer. For example, in the case of coating on a medical device, there occur no problems such as a peeling of the coating layer, and a drug is released at an adequate rate.

—Drug

A drug component, which is contained in the controlled drug-release composition of the present invention and is the target of controlled release, is generally a drug including a pharmaceutical and a quasi drug, but a drug may be a cosmetic, an agricultural chemical in addition to a pharmaceutical according to the application and objective.

The target drug is not particularly limited so long as it is dissolved in an organic solvent which dissolves the above-mentioned organic polymeric material. Therefore, the drug is selected arbitrarily according to the target therapeutic effect and drug action, and a suitable optional bioactive drug may be an object of the present invention. In addition, the drug is not limited to one type but may be used in a form where multiple drugs are allowed to coexist. For example, in two-, three- or four-drug combination therapy adopted for treatment of gastric ulcer, tuberculosis, common cold and the like, multiple drugs are used simultaneously to ensure the synergistic effect and/or complementary action by the combination.

The drug is specifically exemplified by an anticoagulant drug (for example, synthetic anticoagulant drug, antiplatelet drug, antithrombin agent), a hemostatic drug, an angiogenesis inhibitor, capillary stabilizer, an antiproliferative agent for preventing blood vessel restenosis, an antithrombotic agent or a wound treating agent or the like.

In addition, there may be mentioned an anticancer drug, an immune-suppressing drug, an antipyretic analgesic drug, anti-inflammatory drug, a cough suppressant and expectorant, an antiulcer drug, sedative drug, a muscle relaxant, an antidepressant drug, an antiepileptic drug, an antituberculous drug, an antiarrhythmic drug, a vasodilator drug, a cardiac stimulant, an antiallergic drug, an antihypertensive and a diuretic drug, a diabetes therapeutic agent, a hormone preparation, a bioactive peptides, narcotic antagonist, bone resorption inhibitor, antirheumatic drug, an antifertility drug, a hepatic drug, a stomachic digestive, an antiflatulent, vitamin preparation, a vaccine preparation, a constipation drug, a hemorrhoid drug, various enzyme preparations, an antiprotozoal drug, an interferon inducer, an anthelmintic agent, an antimicrobial for external use, a parasitic skin disease drug, a contrast agent and the like.

Further, the specifically applicable drugs are exemplified as follows, but the present invention is not limited to these exemplifications. In addition, the drug may be in a form of a salt or a derivative in addition to itself.

The anticoagulant drug includes heparin sodium, sodium citrate and the like. In addition, as a low-molecular-weight synthetic anticoagulant drug, argatroban which is an antithrombin drug and sarpogrelate hydrochloride which is an antiplatelet drug exhibit blood compatibility. Further, the angiogenesis inhibitor includes fumagillin, a fumagilol derivative, angiogenesis inhibiting steroid and the like. The hemostatic drug includes thrombin, thromboplastin, acetomenaphthone, menadione sodium bisulfite, tranexamic acid, ε-aminocaproic acid, adrenochrom monoaminoguanidine methane sulfonate, carbazochrome sodium sulfonate and the like.

The antitumor agent includes methotrexate, actinomycin D, mitomycin D, bleomycin hydrochloride, daunorubicin hydrochloride, vinblastine sulfate, vincristine sulfate, adriamycin, neocarzinostatin, fluorouracil, cytosine arabinoside, Krestin, Picibanil, Lentinan, Bestatin, levamizole, azimexon, glycyrrhizin, cisplastin, paclitaxel and the like.

The immune-suppressing drug includes rapamycin, ciclosporin, tacrolimus, methotrexate, azathioprine, cyclophosphamide, adrenocorticosteroid (for example, dexamethasone), mizoribine and the like.

The antibiotic includes tetracycline hydrochloride, oxytetracycline hydrochloride, doxycycline hydrochloride, rolitetracycline, streptomycin, novabioxin, neomycin, erythromycin, colistin, lincomycin, salinomycin, nigericin, kanamycin, kitasamycin, tylosin, furaltadone, vancomycin, spiramycin, ristocetin, soymacin, amikacin, fradiomycin, sisomycin, gentamicin, kanendomycin, dibekacin hydrochloride, lividomycin, tobramycin, ampicillin, amoxicillin, ticarcillin, piperacillin, cephaloridine, cephalothin, cefsulodin, cefotiam, cefinenoxime, cefmetazole, cefazolin, cefotaxime, cefoperazone, ceftizoxime, moxolactan, sulfazecin, azthreonam, thienamycin, metronidazole, clarithromycin and the like.

The antipyretic analgesic drug includes sodium salicylate, sulpyrine, diclofenac sodium, sodium flufenamate, indomethacin sodium, morphine hydrochloride, pethidine hydrochloride, oxymorphane, levorphanol tartrate and the like.

The cough suppressant and expectorant includes ephedrine hydrochloride, methylephedrine hydrochloride, noscapine hydrochloride, codeine phosphate, dihydrocodeine phosphate, chlophedianol hydrochloride, aroclamide hydrochloride, picoperidamine hydrochloride, cloperastine, isoproterenol hydrochloride, protokylol hydrochloride, salbutamol sulfate, terbutaline sulfate and the like.

The antiulcer drug includes histidine hydrochloride, metoclopramide and the like. The sedative drug includes prochlorperazine, chlorpromazine hydrochloride, trifloperazine, atropine sulfate, methylscopolamine bromide and the like. The muscle relaxant includes pancuronium bromide, tubocurarine chloride, pridinol methanesulfonate and the like. The antidepressant drug includes imipramine, clomipramine, noxiptiline, phenelzine sulfate and the like. The antiepileptic drug includes chlordiazepoxide hydrochloride, acetazolamide sodium, phenytoin sodium, ethosuximide and the like.

The diabetes therapeutic agent includes phenformin hydrochloride, glymidine sodium, glipizide and the like. The antituberculous drug includes sodium paraminosalicylate, ethambutol, isoniazid. The antiarrhythmic drug includes propranol hydrochloride, alprenolol hydrochloride, bufetolol hydrochloride, oxprenolol hydrochloride and the like. The vasodilator drug includes diltiazem hydrochloride, oxyfedrine hydrochloride, tolazoline hydrochloride, hexobendine, bamethan sulfate and the like. The cardiac stimulant includes aminophylline, theophyllol, etilefrine hydrochloride, transbioxocamphor and the like. The antiallergic drug includes chlorpheniramine maleate, methoxyphenamine hydrochloride, diphenhydramine hydrochloride, tripelennamine hydrochloride, methdilazine hydrochloride, clemizole hydrochloride, methoxyphenamine hydrochloride, diphenylpyraline hydrochloride and the like. The antihypertensive and diuretic drug includes pentolinium, hexamethonium bromide, mecamylamine hydrochloride, ecarazine hydrochloride, clonidine hydrochloride and the like.

The hormone preparation includes prednisolone sodium phosphate, prednisolone sucinate, sodium dexamethasone sulfate, betamethasone sodium phosphate, hexestrol diacetate, hexestrol diphosphate, methimazole and the like.

The narcotic antagonist includes nalorphine hydrochloride, naloxone hydrochloride, levallorphan tartrate and the like. The bone resorption inhibitor includes (sulfur-containing alkyl)aminomethylene bis-phosphonic acid and the like.

The bioactive peptides may be either an oligopeptide or a polypeptide and are not particularly limited so long as it has bioactivity. Those having a molecular weight of approximately 200 to 80,000 are preferred. Specific examples include luteinizing hormone-releasing hormone and its derivative, insulin, somatostatin or its derivative, growth hormone, prolactin, adenocorticotropic hormone, thyrotropic hormone, melanocyte-stimulating hormone, parathyroid hormone, vasopressin, oxytocin, calcitonin, glucagon, gastrin, secretin, cholecystokinin, pancreozymin, angiotensin, enkephalin, protein synthesis stimulating peptide, human chorionic gonadotropin, human placental lactogen, luteinizing hormone, follicle stimulating hormone, various types of interferon, interleukin, endorphin, kyotorphin, tuftsin, thymopoietin, thymosin, thymostimulin, thymic humoral factor, tumor necrosis factor, colony-inducing factor, nerve growth factor, Substance P, kallikrein, motilin, dynorphin, bombesin, cerulein, bradykinin, asparaginase, urokinase, lysozyme chloride, Polymyxin B, colistin, gramicidin, bacitracin, erythropoietin, platelet-derived growth factor, growth hormone-releasing factor, epidermal growth factor, and the like.

The contrast agent includes an iodine-based X-ray contrast agent (iodixanol, iopamidol, iotrolan and the like), an MRI contrast agent (gadolinium compounds), an untrasonic contrast agent (Echovist, Levovist and the like), a near infrared fluorescent contrast agent (indocyanine-based compounds) and the like.

In addition to medical drugs, it may be various cosmetics (such as creams, emulsions, packs, mascras, pilatories, skin whitening agents and the like), agricultural chemicals (such as antibacterial agents, herbicides, insecticides and the like. Specific examples are described in Japanese Patent Laid-Open Publication No. H07-330629), and drugs such as auxins, plant hormones, insect hormones and the like.

Since setting the release rate of a drug is based on the minimum concentration for manifestation of the pharmacological effect in the blood or tissue, it should be studied by individual drug. Similarly, in setting the length of the release period, it is required to consider the information and clinical conditions of an individual patient, the object of treatment, the treatment details and the like. Therefore, the additive amount of a drug cannot be determined arbitrarily, however, in consideration of the balance between the pharmacological effect and cost, it is in the range of typically from 1 to 150 parts by weight, preferably from 1 to 70 parts by weight, more preferably from 5 to 70 parts by weight and especially preferably from 10 to 60 parts by weight, based on 100 parts by weight of the above-mentioned organic polymeric material. When the additive amount is within the above range, it is preferable because the pharmacological effect may be maximized while minimizing the concern of the solubility and side-effect of a drug.

—Other Additives

The controlled drug-release composition of the present invention comprises the above-mentioned organic polymeric material, drug and release auxiliary agent, and is applied to the medical devices as described later. The present composition may further comprise a cell adhesion material or an endothelialization promoting agent on a surface of a medical device where necessary.

The cell adhesion material is exemplified by collagen, fibronectin, vitronectin, laminin and the like.

In the case where the endothelialization promoting agent is applied to the following medical devices, especially, a stent which, described later, is used for a blood vessel system, it promotes the migration, settlement and proliferation of an endothelial cell on its surface at a relatively early stage after indwelling treatment. Such an endothelialization promoting agent is exemplified by a cell adhesion oligopeptide and the like.

The endothelial cell covering the innermost layer of the vascular intima not only plays a role of lining the vascular inner wall but also exhibits various functions such as antithrombogenicity, homeostasis of the blood vessel and blood flow such as repair and the like, angiogenesis, production and secretion of various factors and regulation substances. The vascular endothelial cell engages not only in the healing process for the damages of the vascular inner wall but also in so-called angiogenesis, and in either case, it goes through a process in which there occur the translocation, migration and settlement of biological ingredients such as protein, macrophage and the like to the damaged sites and subsequently there occur migration, settlement and proliferation of a smooth muscle cell and an endothelial cell.

When attention is paid to such behavior of the endothelial cell, for the avoidance from the foreign substance recognition activity of a living body to a stent which is a main cause for restenosis and reocclusion, it is worth to consider the migration, settlement and proliferation of the vascular endothelial cell on the stent surface at a relatively early stage after treatment. The endothelial cell settled on the stent surface proliferates to cover the stent in a monolayer. When such endothelialization occurs, it is considered that a so-called pseudo-state as in the vascular inner wall is rapidly formed on a stent, the stent is unlikely to be targeted by the foreign substance recognition of a living body, and thus the immune and foreign substance exclusion functions do not work. In other words, there is unlikely to occur the migration of a monocyte or a macrophage, as a causative agent for an inflammatory reaction, to the stent-indwelling site. In order to promote the endothelialization of the stent surface, the endothelialization promoting agent is preferably contained in the above-mentioned composition.

In addition, the composition may comprise a binder, a solubilizer, an emulsifier, a stabilizer and the like that are commonly used, in pharmaceutical technology where necessary. The selection and amount of the auxiliary agent and additives used in formulation are determined arbitrarily according to the above-mentioned organic polymeric material and drug, and medical device to which the present composition is applied.

Drug-Releasable Medical Device

With the advancement of medical engineering technology, there has been studied a technique for achieving a desired purpose by incorporating, implanting, embedding or indwelling some medical tools, devices and equipment inside or outside of a living body mainly for the sake of diagnosis and treatment. The drug-releasable medical device of the present invention relating to such technology, contacts a living body, or is incorporated or indwelled in a living body while retaining the above-mentioned composition. The drug-releasable medical device containing the above-mentioned composition is not particularly limited. The target of the device to which the above-mentioned composition is applied generally corresponds to a medical device used in the medical field, but actually responds to the individual necessity in the medical site.

The term “medical device” herein includes a so-called medical tool. Specific examples include various catheters and drip infusion sets which are used for connection of the outside and inside of the body; a stent, a clip, a stapler, a hemostatic agent, a suture, a fracture fixing device, a pacemaker, an organ replacement medical device (artificial blood vessel, artificial trachea, prosthetic valve, intraocular lens, artificial bone, artificial joint and the like), an artificial organ (artificial skin, artificial breast, artificial lung, artificial heart and the like) which are completely used within the body; an wound covering material, a contact lens, an inlay, an artificial dental root, a dental crown, a denture basal seat, a composite resin for repair, a GTR material for dental surgery and the like which are used in the vicinity of the body surface. In addition, a biosensor (for example, a capsule endoscope as a capsule-type sensor), an embedded-type radiation source and the like are included.

The drug-releasable medical device of the present invention contains the controlled drug-release composition of the present invention, thereby releasing the drug at a predetermined site in the body. That is, when the drug-releasable medical device is incorporated, implanted or indwelled at a predetermined site in the body or at a body surface site, the drug which has been retained is released, with the release timing and release rate, and the release amount and the release period being under control. The retention form varies depending on the type, application and the like of the medical device, but is not particularly limited, and for example, there may be used various application forms such as application, coating including spraying, inclusion in pores, cohesion, binding, adhesion and fixing, winding of a film, a tape and the like for drug delivery carrying the present composition. The most convenient form is to form a layer of the above-mentioned composition on a surface of a medical device and may be widely used because the surface is changed to the functional surface.

As a method of retaining the controlled drug-release composition of the present invention on the target medical device, the controlled drug-release composition is adhered and fixed on the surface of a medical device in a form of layer by immersing the medical device in the composition solution and then removing the solvent, or by spraying the solution on the medical device surface and then removing the solvent, or by applying the solution on the medical device and then removing the solvent. In the case where such coating is provided on a medical device, the coating layer has a thickness of one to thousands nanometers (nm) and preferably tens to hundreds nm, and the thinner the coating layer, the less tendency of peeling off. In the case where the composition of the present invention is singly embedded in a medical device, the thickness is not particularly a problem, and in addition, there may be selected an arbitrary shape such as a sheet shape, a spherical shape, a bar shape and the like.

The drug-releasable medical device of the present invention is not wholly shown because the application forms varies according to the medical device, but the representative example includes the application to the surgery field including a stent, or dental treatment including an inlay. Especially preferred is a stent, a catheter, a clip, a capsule sensor, an organ replacement medical device or an artificial organ.

### BEST MODE FOR CARRYING OUT THE INVENTION

A stent is referred to as a medical device to which the controlled drug-release composition of the present invention is suitably applied. Therefore, hereinafter, there is explained the provision of a drug sustained-release property to a stent and such a stent, as an aspect in which the present invention is specifically applied.

Many angioplasties are performed as a treatment method of coronary artery occlusion, which is a main cause of myocardial infarction. This method is based on securement of blood vessel flow chiefly by balloon dilation and angioplasty by laser excision, and many favorable treatment results have been reported. On the other hand, it has been reported that restenosis and reocclusion of the blood vessel after treatment occurred at a high percentage of 40 to 50%, which was a problem of this method.

For the physical problems of restenosis and reocclusion, there have been tried drug administration, reinsertion and redilation of a balloon catheter or laser treatment and the like. However, it is hard to say that any of them are an ultimate solution and they compel much pain and burden to patients. Consequently, a stent indwelled inside the blood vessel is used. The stent revises and supplements the incised portion as well as prevents shrinkage of the blood vessel, thereby effectively reducing the incidence of restenosis of artery occlusion patients.

A stent for the blood vessel is a small member of a tubular medical device made of a metal material or a polymeric material. A typical treatment method of the representative occluded blood vessel using a stent is as follows. The stent for the blood vessel is indwelled in the occluded blood vessel portion through a balloon catheter inserted in the blood vessel lumen. Thereafter, the patency of the blood vessel is secured by irreversibly enlarging the diameter of the stent by dilating the balloon, or by self-dilating the stent by any of means such as a magnetic induction type heating after indwelling the stent in the arterial vessel. In this way, the stent maintains good blood flow over a long period of time.

There have been conventionally made various proposals concerning the material, shape and application of a stent for the purpose of treatment of arterial vessel occlusion including coronary artery occlusion. However, since conventional materials cannot still completely avoid the risk of restenosis and reocclusion, this is a bottleneck for the application of angioplasty using a stent. Therefore, a stent with less possibility of restenosis and reocclusion has been desired from the medical site. In the case where a drug is applied to a stent as a preferred embodiment of the above-mentioned drug-releasable medical device, there are used a synthetic anticoagulant drugs an anticancer drug, an immune-suppressing agent and the like. In order to provide a stent with a drug release function, especially a drug sustained-release property, for example, there may be any methods such as a method of applying (coating, embedding and the like) a composition containing a drug on the stent surface (1) and a method of coating a carrier carrying a drug released or gradually released and further an release auxiliary agent if necessary on the stent surface (2). In the embodiment (1), there may be preferably used the above-mentioned controlled drug-release composition comprising an organic polymeric material and a drug. Further, in the embodiment (2), a polymeric material covering the stent surface carries a drug to be gradually released.

As the specific synthetic anticoagulant drug, sarpogrelate hydrochloride, argatroban and the like are preferred. A composition comprising such drugs is coated on the surface of a stent as a coating layer. The drug is gradually released in the blood or to blood vessel wall from the surface of such a drug releasable stent at a desired release rate. Since the composition of the present invention releases a drug at a high rate, a sufficient amount of an anticoagulant drug is released to exhibit the pharmacological effect from the initial time of indwelling of a stent.

The stent may have any structure, shape, material, size or embodiment so long as it has the above-mentioned characteristics. Various uses and applications of the present invention are easy for those skilled in the art. Therefore, the above-mentioned stent may be applied to all aspects for the purpose of preventing restenosis and reocclusion of vasa (blood vessel, lymphatic vessel, bile duct, ureter, trachea and the like).

The stent of the present invention is characterized in that argatroban (an antithrombin drug) or sarpogrelate hydrochloride (an antiplatelet drug) or both drugs are gradually released from the surface. Preferably, the drug to be released gradually is carried in a polymeric material coated on the surface of a metal forming the stent or in a porous stent substrate. The stent of the present invention is preferably used for treating coronary artery stenosis.

Stent

The material and structure of the stent of the present invention may be practically of any design as long as the following surface treatment is provided. This means that the present invention may prevent restenosis and reocclusion from occurring while retaining the characteristics and functions of various stents.

The stent is the one which is not changed in shape before and after inserting in the blood vessel, or may be a balloon dilation type, a self-dilation type and their combination. For the stent relating to the present invention, any material may be suitably used as long as it has physical properties capable of performing the design. Specifically, the metal material may be exemplified by stainless, cobalt-chrome alloy, tantalum, titanium, tungsten, platinum, cobalt, and alloy thereof, or the like.

In the case of using a material other than metal, a material which may carry an anticoagulant drug is preferred in order to meet the object of the present invention as mentioned later. The polymeric material meeting such requirements includes PET (poly(ethyleneterephthalate)), PBT (poly(butyleneterephthalate)), poly(carbonate), poly(ethylene), poly(propylene), poly(acetal), poly(styrene) and the like. The biodegradable polymer may be exemplified by poly(lactic acid), poly(glycolic acid), poly(malic acid) and a copolymer thereof, poly(hydroxyl esters) such as poly(caprolactone) and the like.

As the stent of the present invention, a metal material is especially preferable, and the shape may be cylindrical, an accordion shape, a structure having bent portions, a mesh shape and a wire shape as a solid molded product, and materials of various shapes may be basically used when they cause no problems with the strength after indwelling in the blood vessel and physical damaging properties to the blood vessel wall.

Anticoagulant Drug

At least one of argatroban (an antithrombin drug) and sarpogrelate hydrochloride (an antiplatelet drug) is gradually released from the surface of the stent of the present invention. In order to realize such release, argatroban or sarpogrelate hydrochloride or both of these synthetic anticoagulant drugs are carried in a polymeric material coated on the surface of a metal forming the stent.

As one of the drugs for suppressing blood coagulation, argatroban, which is an antithrombin drug used in the present invention, is an arginine derivative-based synthetic antithrombin drug having a chemical structure represented by the following formula. Three-leg structure of argatroban is sterically bonded to the active site of thrombin to strongly inhibit the main function of thrombin, that is, the generation of fibrin, the stabilization of fibrin by the activation of factor XIII and the platelet coagulation, thereby exhibiting antithrombin action. In this way, since argatroban directly acts on thrombin, it has smaller individual difference than that of heparin and the action is secure and the action exertion is rapid. In addition, since argatroban may not be inhibited by a naturally occurring substance, and has a low molecular weight, it may act on fibrin-bonded thrombin and securely prevent the growth of thrombus. Further, argatroban responds to white thrombus which is formed under high shear stress and is not prevented by heparin, and suppresses the white thrombus.

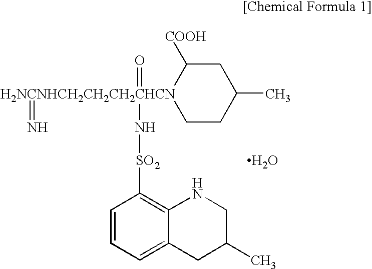

Sarpogrelate hydrochloride, which is another anticoagulant drug used in the present invention, has a function of suppressing platelet activation, and the action mechanism is considered as follows.

Serotonin (5-HT), which is released by the activated platelet adhered and coagulated at a damaged site of vascular endothelium, has various pharmacological actions, i.e. enhances the coagulation of platelet at the damaged site through 5-HT₂ receptor on platelet membrane and vascular smooth muscle cell membrane, and causes the constriction of the blood vessel at the damaged site as well as proliferates vascular smooth muscle cells, thereby bringing about peripheral circulatory failure. Anplag® (sarpogrelat) exhibits suppressing action on platelet coagulation, especially the platelet coagulation enhanced by serotonin, and vasoconstriction inhibitory action by selectively blocking a 5-HT₂ receptor. For this reason, sarpogrelate hydrochloride exhibits efficacy for various thrombus models including chronic arterial occlusion.

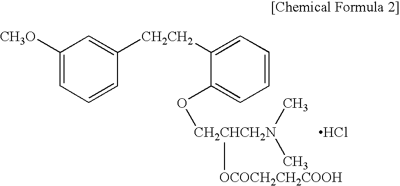

These two drugs are extremely effective especially for suppression of initial thrombus formation, and the oral administration and intravenous administration are widely clinically applied as an anticoagulant therapy. On the other hand, in a short period within a few months immediately after the indwelling of a stent, occlusion prevention of coronary artery by thrombus formation at the indwelling site is a major problem. Therefore, in the case where blood vessel and the like are treated with the stent of the present invention, occlusion by thrombus formation may be effectively prevented by gradually releasing these anticoagulant drugs from the stent. As a result, it is expected to suppress effectively restenosis and reocclusion of blood vessel and the like at the stent indwelling site.

As the medical device provided with antithrombogenicity for gradually releasing argatroban, as mentioned above, a catheter is disclosed in Japanese Patent Laid-Open Publication No. H06-292711 and Japanese Patent Laid-Open Publication No. H06-292718. The former describes that argatroban is melt-kneaded in a thermoplastic polymeric material to be molded into a catheter tube, and the latter describes a method in which a catheter tube is immersed in an organic solvent in which argatroban is dissolved to penetrate argatroban into the tube. In these techniques, the catheter substrate is basically made of a material excellent in mechanical strength and moldability, and as the preferred material which may be used, there is proposed a crystalline thermoplastic elastomer such as segmented nylon, segmented polyurethane, segmented polyester and the like.

—Supporting of Drug in Stent

The drug releasable type stent of the present invention carries the above-mentioned anticoagulant drug, and when it is indwelled inside the predetermined blood vessel, the carried drug is released over a certain fixed period of time. For the method for carrying the above-mentioned polymer containing the drug on a stent, it is not particularly limited and various carrying forms are feasible, but preferred is an application form in which the release timing, the release rate, and the release amount and period may be controlled. For example, there may be mentioned; a method of providing fine pores on a surface of a metal forming a stent by laser ablation, plasma etching or the like and sealing a drug in the pores; a method of forming a stent with a porous metal or a porous inorganic material and sealing a drug in the porous portion; a method of forming a polymeric layer containing a drug on a surface of a metal forming a stent; a method of preparing a stent itself with a polymer containing a drug; a method of winding a film, a tape or the like carrying a drug for drug delivery on a stent; and the like. Among these, the method forming a layer of the drug-containing polymer on the surface of a metal stent is more convenient. The method may be widely applied since the current stent technique may be used and the stent surface is changed to the functional surface as is. Therefore, the method is preferable.

In the case where the surface of the metal forming the above-mentioned stent is a porous body, the drug to be gradually released is dispersed in a polymer and the resulting polymer is carried on the pore portion of the porous body. A drug may be released at a desired release rate and continuously by controlling the pore diameter of a porous stent substrate and retaining a polymeric material carrying the drug in the pore. The porous stent substrate has a pore diameter of preferably 0.01 nm to 300 nm and more preferably 0.1 nm to 100 nm.

Polymeric Material for Supporting Used for Coating of Stent

The present inventors have found that the compatibility of a drug and a polymer becomes an important factor for a polymeric material carrying the drug in order to gradually release the anticoagulant drug for a fixed period of time and the drug is more desirably carried on an amorphous polymer. In addition, a material having a glass transition point of 37° C. which is a body temperature, or lower is preferred. In the case of indwelling a material having a glass transition point of 37° C. or lower, in the blood vessel, the temperature of the polymer reaches the glass transition point or higher, thereby increasing the molecular mobility of the main chain and promoting the drug release. In the case of using a crystalline polymer, a polymer crystal phase and a drug phase are clearly phase-separated thereby sometimes causing a phenomenon that the drug is segregated to the surface. For this reason, a drug is released at once, that is, a so-called bursty release occurs and subsequently the drug release is significantly decreased.

On the other hand, the anticoagulant drug used in the present invention has hydrophilicity in that it has a basic group or an ionic group as shown by the chemical structural formula, but has relatively high lipophilicity, has significantly low solubility in water and rather has high solubility in alcohol. Therefore, the anticoagulant drug has poor compatibility with a polymeric material having extremely high hydrophobicity such as polyolefin, it is predicted that bursty release by phase separation occurs and the subsequent release rate is significantly reduced with these materials, as with a crystalline polymeric material. For example, in a (meth)acrylate-based polymeric material, the ester residue preferably is an ester having 4 or less carbon atoms, e.g. methyl ester, ethyl ester, propyl ester and butyl ester, or an alkyl ester having a hydroxyl group, an alkoxyl group, an ethylene oxide ether group (—(CH₂CH₂O)—) which may exhibit hydrophilicity.

As the preferred amorphous polymeric materials for coating in the present invention, there may be mentioned, but not limited to, a poly(alkylmethacrylate), a poly((hydroxyalkyl)methacrylate) and a copolymer thereof such as poly(butylmethacrylate), poly(ethylmethacrylate), poly(propylmethacrylate), poly(hydroxyethylmethacrylate); a poly(alkylacrylate) or its copolymer such as poly(butylacrylate), poly(ethylacrylate), poly(propylacrylate), poly(methoxyethylacrylate); an aliphatic poly(carbonate) and its copolymer such as poly(butylenecarbonate), poly(ethylenecarbonate); a polyvinyl compound and its copolymer such as poly(vinyl acetate), poly(vinylpirolidone), partially saponified poly(vinylalcohol), poly(vinylether); a biodegradable polymer containing lactic acid or glycolic acid as an ingredient, DL-poly(lactic acid), DL-lactic acid-glycolic acid copolymer, and the like.

The above-mentioned amorphous polymeric material, in contrast to a crystalline polymer, is excellent in solubility into an organic solvent, and many organic solvents become a target for use in coating a stent, thus increasing the technical convenience.

Release Auxiliary Agent

As mentioned above, when a release auxiliary agent promoting the drug release is added to a polymeric material forming a DDS matrix, a sustained-release rate of a drug is increased, and this is no exception in the case of a stent. Therefore, in the stent of the present invention, in the case where a desired drug release rate is not obtained when the above-mentioned polymeric material is combined in applying the drug, a targeted release rate may be obtained by using an auxiliary agent. The auxiliary agent is especially effective for a polymeric material having a glass transition point higher than the body temperature. In the case of a biodegradable polymeric material such as poly(lactic acid), a lactic acid-glycolic acid copolymer, the addition of an auxiliary agent is effective because the glass transition point is decreased. The auxiliary agent is basically lipid-soluble, but a low molecular weight substance exhibiting a certain degree of water solubility is preferred. The reason is because it is involved with a problem of compatibility to both a polymer and a drug. A long-chain aliphatic ester and the like which are poor in hydrophilicity, is not preferable because it is poor in compatibility with the drug. In addition, a low-molecular-weight compound like glycerin, which is water soluble and has extremely low lipophilicity, is also not preferable because it is poor in compatibility with a polymeric material and the drug. As a preferred auxiliary agent in the present invention, there may be mentioned an ester of an organic acid selected from citric acid, tartaric acid or malic acid, or a diester and a monoester of glycerin (for example, monoacetin, diacetin), and the specific examples are esters exemplified above.

These auxiliary agents may be used singly or in combination of two or more kinds. The additive amount may be set arbitrarily according to the drug release rate, but is generally in the range of from 5 to 60% by weight and preferably from 10 to 60% by weight, based on the weight of a polymeric material. When the additive amount is within this range, a good addition effect is obtained and the coating layer exhibits sufficient mechanical strength and is unlikely to be peeled off from a stent surface.

Drug-Containing Coat Layer

As a method for forming a polymer layer containing a drug, that is, a layer of the above-mentioned polymer containing a drug on a stent surface, there may be mentioned an application method of applying a solution obtained by dissolving a drug, a polymeric material, and other additives including an release auxiliary agent if needed and the like in a common solvent which dissolves them to the stent surface; an immersion method of immersing a stent in said solution and then drawing up and drying; a spray-coat method of spraying the solution on the stent surface to coat on the stent; and the like. Among these, a method in which coating may be performed suitably is the immersion method, and according to this method, coating may be conveniently performed both on the inner surface and outer surface of a stent. Especially in the case of adequately performing the coating treatment of the stent inner surface, which is also a blood-contacting surface, sufficient functions are frequently provided for imparting antithrombogenicity and reducing arterial vessel reocclusion.

The coating layer formed preferably has a thickness between 0.05 μm to 30 μm. When the thickness is within this range, a sufficient amount of drug is carried and thus the drug release is secured for a targeted period of time, and the coating layer exhibits a good followability to the deformation of stent accompanied by heart beating and is less likely to be cracked or peeled off.

Drug Carrying Amount

The drug-carrying amount in the stent of the present invention is determined by the desired release rate and the desired period of drug release duration. Since a bursty, large amount of release for a short time results in depletion of a drug in a short period of time, it is essential to avoid this. The period of the release duration preferably is a few weeks to a few months of the sustained release, from the viewpoint of preventing the initial thrombus formation. Therefore, for the release rate of a drug at the time of three weeks (21 days) after indwelling the stent, in either argatroban or sarpogrelate hydrochloride, the elution rate preferably is 1×10⁻³ μg/mm²-h to 1 μg/mm²-h, and more preferably 1×10⁻³ μg/mm²-h to 0.5 μg/mm²-h. When the elution rate is within this range, it is preferable because the anticoagulation activity is maintained for long period of time.

The upper limit of the release rate of a drug is not particularly limited so long as it does not exceed the toxic level. However, in view of both matters that the carrying amount of a drug on a stent is limited and the maximum amount is considered to be a few hundreds μg, and that the release is desired to be continued at least approximately 40 days, the maximum rate is considered to be substantially approximately 1 μg/mm²-h. For example, the elution rate of argatroban from a catheter is considered to be approximately 1.0×10⁻⁴ to 1.0×10⁻¹ μg/cm²-min and preferably 2.5×10⁻⁴ to 7.0×10⁻³ μg/cm²-min (Artificial Organs 14 (2), p 679-682 (1985)). In consideration of both the clinical findings of the effective pharmacological concentration in blood of argatroban and sarpogrelate hydrochloride and the above findings, it is reasonable to consider that the elution rate of argatroban and sarpogrelate hydrochloride in the present invention is in the range described in the above-mentioned literature “Artificial Organs”. However, the above published document states the release rate for a catheter on the premise of short-term indwelling. In contrast, the stent of the present invention is a permanent indwelling, and therefore, it is essential to prevent thrombus formation over at least approximately three weeks immediately after indwelling. When this period is passed, the regeneration of endothelial cell progresses and the risk of thrombus formation is significantly reduced. Therefore, it is critically important to maintain the release rate at the time when approximately three weeks has passed after indwelling, in addition to the release rate immediately after indwelling.

### EXAMPLES

The present invention is explained in further detail by the following examples, but these examples are not intended to restrict the present invention. The numerical conditions, treating methods and the like used in the examples, such as material, use amount, concentration, treating time, treating temperature and the like are only preferred examples within the scope of the present invention.

### Examples 1 to 3 and Comparative Examples 1 to 3

As shown in Table 1, a solution was prepared by dissolving 90 mg of poly(lactic acid) or 90 mg of a lactic acid-glycolic acid copolymer, 10 mg of triethyl citrate as a release auxiliary agent and 10 mg of sarpogrelate hydrochloride of an antiplatelet drug in 1 mL of hexafluoroisopropanol. The resulting solution was cast on a glass Petri dish having a diameter of 41 mm and was air-dried to obtain a drug-carrying composition. The elution amount of the drug was pursued by immersing the composition in 100 mL of a phosphate buffer solution having a pH of 7.4 and sampling a portion of the buffer solution periodically and subsequently measuring the absorbance (Abs) at 270 nm which is a characteristic absorption band of sarpogrelate hydrochloride. The absorbance after three weeks from the start of elution is shown in Table 1.

As Comparative Examples 1 to 3, the similar elution experiments were carried out under the same conditions as in Examples except that the release auxiliary agent was not added.

TABLE 1

| | Polymer | | |
|---|---|---|---|
| | (Mole Ratio of | | |
| | Lactic | Release | Amount |
| | Acid/Glycolic | Auxiliary Agent | Released |
| | Acid) | (10 mg) | (Abs) |
| Example 1 | 100/0 | Added | 0.173 |
| Comparative | 100/0 | Not Added | 0.013 |
| Example 1 | | | |
| Example 2 | 85/15 | Added | 0.120 |
| Comparative | 85/15 | Not Added | 0.027 |
| Example 2 | | | |
| Example 3 | 50/50 | Added | 0.067 |
| Comparative | 50/50 | Not Added | 0.002 |
| Example 3 | | | |

### Examples 4 to 6 and Comparative Example 4

A solution was prepared by dissolving 90 mg of a lactic acid/glycolic acid (50/50) copolymer, 10 mg of dialkyl tartrate as a release auxiliary agent and 10 mg of sarpogrelate hydrochloride of an antiplatelet drug in 1 mL of hexafluoroisopropanol. The resulting solution was cast on a glass Petri dish having a diameter of 41 mm and was air-dried to obtain a drug-carrying composition. The elution amount of the drug was pursued by immersing the composition in 100 mL of a phosphate buffer solution having a pH of 7.4 and sampling a portion of the buffer solution periodically and subsequently measuring the absorbance (Abs) at 270 nm which is a characteristic absorption band of sarpogrelate hydrochloride. The absorbance after three weeks from the start of elution is shown in Table 2.

TABLE 2

| | Release Auxiliary | Amount |
|---|---|---|
| | Agent | Released (Abs) |
| Example 4 | Dimethyl Tartrate | 0.033 |
| Example 5 | Diethyl Tartrate | 0.007 |
| Example 6 | Diisopropyl Tartrate | 0.003 |
| Comparative | Not added | 0.002 |
| Example 4 | | |

### Examples 7 to 10 and Comparative Examples 5 and 6

As shown in Table 3, a solution was prepared by dissolving 90 mg of poly(lactic acid) or 90 mg of a lactic acid-glycolic acid copolymer, 10 mg of diethyl tartrate or triethyl citrate as a release auxiliary agent and 10 mg of argatroban of an antithrombin drug in 1 mL of hexafluoroisopropanol. The resulting solution was cast on a glass Petri dish having a diameter of 41 mm and was air-dried to obtain a drug-carrying composition. The elution amount of the drug was pursued by immersing the composition in 100 mL of a phosphate buffer solution having a pH of 7.4 and sampling a portion of the buffer solution periodically and subsequently measuring the absorbance (Abs) at 330 nm which is a characteristic absorption band of argatroban. The absorbance after three weeks from the start of elution is shown in Table 3.

As Comparative Examples 5 and 6, the similar elution experiments were carried out under the same conditions as in Examples except that the release auxiliary agent was not added.

TABLE 3

| | Polymer (90 mg) | | |
|---|---|---|---|
| | (Mole Ratio of | | |
| | Lactic | Release | Amount |
| | Acid/Glycolic | Auxiliary Agent | Released |
| | Acid) | (10 mg) | (Abs) |
| Example 7 | 100/0 | Diethyl Tartrate | 0.310 |
| Example 8 | 100/0 | Triethyl Citrate | 0.092 |
| Comparative | 100/0 | Not Added | 0.056 |
| Example 5 | | | |
| Example 9 | 50/50 | Diethyl Tartrat | 0.200 |
| Example 10 | 50/50 | Triethyl Citrate | 0.052 |
| Comparative | 50/50 | Not Added | 0.034 |
| Example 6 | | | |

### Examples 11 to 16 and Comparative Examples 7 and 8

As shown in Table 4, a solution was prepared by dissolving 90 mg of poly(lactic acid) or 90 mg of a lactic acid-glycolic acid copolymer, 10 to 30 mg of diethyl tartrate as a release auxiliary agent and 10 mg of argatroban of an antithrombin drug in 1 mL of hexafluoroisopropanol. The resulting solution was cast on a SUS 316L Petri dish having a diameter of 18 mm and was air-dried to obtain a drug-carrying composition. The elution amount of the drug was pursued by immersing the composition in 50 mL of a phosphate buffer solution having a pH of 7.4 and sampling a portion of the buffer solution periodically and subsequently measuring the absorbance (Abs) at 330 nm which is a characteristic absorption band of argatroban. The absorbance after 7 days from the start of elution is shown in Table 4.

As Comparative Examples 7 and 8, the similar elution experiments were carried out under the same conditions as in Examples except that the release auxiliary agent was not added.

TABLE 4

| | Polymer (90 mg) | | |
|---|---|---|---|
| | (Mole Ratio of | | |
| | Lactic | Diethyl | Amount Released |
| | Acid/Glycolic | Tartrate | after 7 days |
| | Acid) | (mg) | (Abs) |
| Example 11 | 100/0 | 10 | 0.0146 |
| Example 12 | 100/0 | 20 | 0.030 |
| Example 13 | 100/0 | 30 | 0.047 |
| Comparative | 100/0 | Not added | 0.007 |
| Example 7 | | | |
| Example 14 | 50/50 | 10 | 0.012 |
| Example 15 | 50/50 | 20 | 0.017 |
| Example 16 | 50/50 | 30 | 0.010 |
| Comparative | 50/50 | Not added | 0.008 |
| Example 8 | | | |

### Examples 17 to 19

As shown in table 5, a solution was prepared by dissolving 90 mg of poly(lactic acid), 30 mg of diethyl tartrate as a release auxiliary agent and argatroban of an antithrombin drug of the specified amounts shown in Table 5 in 1 mL of hexafluoroisopropanol. 600 μL of the resulting solution was cast on a SUS 316L Petri dish having a diameter of 18 mm and was air-dried to obtain a drug-carrying composition. The elution amount of the drug was pursued by immersing the composition in 50 mL of a phosphate buffer solution having a pH of 7.4 and sampling a portion of the buffer solution periodically and subsequently measuring the absorbance (Abs) at 330 nm which is a characteristic absorption band of argatroban. The absorbance after two weeks from the start of elution is shown in Table 5.

TABLE 5

| | Polymer (90 mg) | | | Amount |
|---|---|---|---|---|
| | (Mole Ratio of | | | Released |
| | Lactic | Diethyl | | after |
| | Acid/Glycolic | Tartrate | Argatroban | 14 days |
| | Acid) | (mg) | (mg) | (Abs) |
| Example 17 | 100/0 | 30 | 20 | 0.10 |
| Example 18 | 100/0 | 30 | 30 | 0.19 |
| Example 19 | 100/0 | 30 | 40 | 0.21 |

### Examples 20 and 21

A solution was prepared by dissolving 90 mg of poly(lactic acid), 30 mg of dimethyl tartrate or diethyl malate as a release auxiliary agent and 30 mg of argatroban of an antithrombin drug in 1 mL of hexafluoroisopropanol. 600 μL of the resulting solution was cast on a SUS 316L Petri dish having a diameter of 18 mm and was air-dried to obtain a drug-carrying composition. The elution amount of the drug was pursued by immersing the composition in 50 mL of a phosphate buffer solution having a pH of 7.4 and sampling a portion of the buffer solution periodically and subsequently measuring the absorbance (Abs) at 330 nm which is a characteristic absorption band of argatroban. The absorbance after two weeks from the start of elution is shown in Table 6.

TABLE 6

| | Release Auxiliary | Amount |
|---|---|---|
| | Agent | Released (Abs) |
| Example 20 | Dimethyl Tartrate | 0.35 |
| Example 21 | Diethyl Malate | 0.16 |

### Examples 22 and 23

A solution was prepared by dissolving 90 mg of poly(lactic acid), 20 mg of monoacetin which is monoacetate of glycerin or 20 mg of diacetin which is diacetate of glycerin as a release auxiliary agent and 20 mg of argatroban of an antithrombin drug in 1 mL of hexafluoroisopropanol. 600 μL of the resulting solution was cast on a SUS 316L Petri dish having a diameter of 18 mm and was air-dried to obtain a drug-carrying composition. The elution amount of the drug was pursued by immersing the composition in 50 mL of a phosphate buffer solution having a pH of 7.4 and sampling a portion of the buffer solution periodically and subsequently measuring the absorbance (Abs) at 330 nm which is a characteristic absorption band of argatroban. The absorbance after three weeks from the start of elution is shown in Table 7.

TABLE 7

| | Release Auxiliary | Amount |
|---|---|---|
| | Agent | Released (Abs) |
| Example 22 | Monoacetin | 0.425 |
| Example 23 | Diacetin | 0.198 |

The release rate in the following Examples is defined as follows. A drug-carrying composition is immersed in a phosphate buffer solution (PBS) having a pH of 7.4 at 37° C. for 21 days consecutively, and the absorbance changes of PBS during this period are observed. A drug elution amount during 24 hours is determined from the difference between the absorbance on the 20^{th} day and the absorbance on the 21^{st} day. The drug elution amount is divided by 24 hours and by the surface area of the carrying composition to obtain a value, which is used as a release rate (unit: μg/(h-mm²)). The surface area of a stent may be determined based on the thickness and developed view obtained by observing a stent under a microscope.

### Examples 24 to 33

A solution was prepared by dissolving 15 mg of argatroban or sarpogrelate hydrochloride and 50 mg of the amorphous polymer shown in Table 8 in 0.6 mL of methanol. The resulting solution was cast in a SUS Petri dish having a diameter of 16 mm and was air-dried and dried in vacuo to obtain a drug-carrying composition. The absorbance was determined by immersing the composition in 50 mL of a phosphate buffer solution having a pH of 7.4 and sampling a portion of the buffer solution periodically and subsequently measuring absorbance at 330 nm that is a characteristic absorption band for argatroban and at 270 nm for sarpogrelate hydrochloride. The release rate was determined by measuring the elution amount using the absorbance. The results are shown in Table 8.

TABLE 8

| | | | Release Rate |
|---|---|---|---|
| Examples | Polymer | Drug | (10⁻³ μg/mm²-h) |
| 24 | PolyMEA | Argatroban | 8.9 |
| 25 | | Sarpogrelate | 11.5 |
| | | Hydrochloride | |
| 26 | PolyHEMA | Argatroban | 16.8 |
| 27 | | Sarpogrelate | 28.2 |
| | | Hydrochloride | |
| 28 | PolyEVE | Argatroban | 3.1 |
| 29 | | Sarpogrelate | 2.9 |
| | | Hydrochloride | |
| 30 | Poly(MEA/HEMA) | Argatroban | 20.2 |
| 31 | | Sarpogrelate | 24.6 |
| | | Hydrochloride | |
| 32 | PolyDnPAAm | Argatroban | 15.9 |
| 33 | | Sarpogrelate | 18.1 |
| | | Hydrochloride | |

PolyMEA: Poly(2-methoxyethylacrylate)
PolyHEMA: Poly(2-hydroxyethylmethacrylate)
PolyEVE: Poly(ethylvinylether)
Poly(MEA/HEMA): 2-methoxyethylacrylate/2-hydroxyethylmethacrylate copolymer
PolyDnPAAm: Poly(N,N-di-n-propylacrylamide)

### Comparative Examples 9 to 14

The release rate of argatroban and sarpogrelate hydrochloride was determined in the same manner as in Example 24 except that crystalline poly(caprolactone), poly (hydroxybutyric acid) and poly(caprolactum) were used in place of the amorphous polymer shown in Example 24. The results are shown in Table 9.

TABLE 9

| Comparative | | | Release Rate |
|---|---|---|---|
| Examples | Polymer | Drug | (10⁻³ μg/mm²-h) |
| 9 | Poly(caprolactone) | Argatroban | 0.04 |
| 10 | | Sarpogrelate | 0.032 |
| | | Hydrochloride | |
| 11 | Poly(hydroxybutyric | Argatroban | 0.011 |
| 12 | acid) | Sarpogrelate | 0.009 |
| | | Hydrochloride | |
| 13 | Poly(caprolactum) | Argatroban | 0.06 |
| 14 | | Sarpogrelate | 0.05 |
| | | Hydrochloride | |

### Examples 34 to 40 and Comparative Examples 15 to 17

A solution was prepared by dissolving 50 mg of poly((DL)lactic acid) or 50 mg of (DL)lactic acid/glycolic acid copolymer exhibiting amorphous nature and shown in Table 10, 15 mg of a release auxiliary agent and 15 mg of argatroban or sarpogrelate hydrochloride in 0.5 mL of hexafluoroisopropanol. The resulting solution was cast on a SUS Petri dish having a diameter of 16 mm and was air-dried and dried in vacuo to obtain a drug-carrying composition. The absorbance was determined by immersing the composition in 50 mL of a phosphate buffer solution having a pH of 7.4 and sampling a portion of the buffer solution periodically and subsequently measuring absorbance at 330 nm that is a characteristic absorption band for argatroban and at 270 nm for sarpogrelate hydrochloride. The release rate was determined by measuring the elution amount using the absorbance. As Comparative Examples, the similar elution experiments were carried out under the same conditions as in Examples 34 to 40 except that the crystalline poly((L) lactic acid) and (L)lactic acid-glycolic acid copolymer (50:50) were used. The results of these Examples and Comparative Examples are shown in table 10.

TABLE 10

| | Polymer (50 | | | |
|---|---|---|---|---|
| | mg) | | Elution | |
| | Lactic | | Auxiliary | Release Rate |
| | acid/Glycolic | | Agent | (10⁻³ |
| Examples | acid Ratio | Drugs | (15 mg) | μg/mm²-h) |
| Example 34 | DL100/0 | Argatroban | Diethyl | 12.0 |
| Example 35 | | Sarpogrelate | Tartrate | 16.2 |
| | | Hydrochloride | | |
| Example 36 | DL50/50 | Argatroban | Diethyl | 7.4 |
| Example 37 | | Sarpogrelate | Tartrate | 3.2 |
| | | Hydrochloride | | |
| Example 38 | DL100/0 | Argatroban | Diisopropyl | 5.5 |
| Example 39 | | Sarpogrelate | Tartrate | 8.9 |
| | | Hydrochloride | | |
| Example 40 | DL100/0 | Argatroban | Dimethyl | 4.2 |
| | | | Malate | |
| Comparative | L100/0 | Argatroban | Diethyl | 0.05 |
| Example 15 | | | Tartrate | |
| Comparative | | Sarpogrelate | | 0.04 |
| Example 16 | | Hydrochloride | | |
| Comparative | L50/50 | Sarpogrelate | Diethyl | 0.07 |
| Example 17 | | Hydrochloride | Tartrate | |

### Example 41

### Stent Indwelling Test

A coating solution was prepared by dissolving 24 mg of argatroban, 24 mg of sarpogrelate hydrochloride, 24 mg of diethyl tartrate and 80 mg of (DL)lactic acid/glycolic acid copolymer (50:50) in 10 mL of hexafluoroisopropanol. A stent made of Co—Cr alloy for coronary artery (diameter: 1.55 mm φ, length: 17.4 mm) was immersed in the coating solution and then 0.6 mg of coating was performed on the stent surface by a dip-coating method. One piece of each of the three coated stents and the three uncoated bare-metal stents was indwelled in the coronary artery of three twelve-month old miniature pigs. After one month later, the pigs were killed and the patency persistence by the stents was evaluated. The patency persistence by the coated stents in the respective three pigs was better than that of the uncoated stents (bare-metal stents) and the suppression effect on stenosis was observed by argatroban and sarpogrelate hydrochloride.

1. A controlled drug-release composition comprising: 100 parts by weight of an organic polymeric material being soluble in an organic solvent and insoluble in water; 5 to 60 parts by weight of a lipid-soluble, low molecular weight release auxiliary agent; and 1 to 70 parts by weight of a drug.

2. The controlled drug-release composition according to claim 1, wherein the organic polymeric material is biodegradable or biocompatible, or both.

3. The controlled drug-release composition according to claim 1, wherein the release auxiliary agent is a carboxylic acid ester, a monoester of glycerin or a diester of glycerin.

4. The controlled drug-release composition according to claim 1, wherein the drug is a pharmaceutical product.

5. The controlled drug-release composition according to claim 4, wherein the pharmaceutical product is an anticoagulant drug, an anticancer drug or an immune-suppressing agent.

6. The controlled drug-release composition according to claim 2, wherein the biodegradable organic polymeric material is an aliphatic polyester or an aliphatic poly(carbonate).

7. The controlled drug-release composition wherein the biodegradable organic polymeric material is a poly(lactic acid), a lactic acid-glycolic acid copolymer, a poly(caprolactone) or a poly(hydroxybutyric acid).

8. The controlled drug-release composition according to claim 1, wherein the release auxiliary agent is an ester of an organic acid selected from a citric acid, a tartaric acid or a malic acid.

9. The controlled drug-release composition according to claim 1, wherein the composition further comprises a cell adhesion material or an endothelialization promoting agent.

10. A drug-releasable medical device retaining the controlled drug-release composition according to claim 1.

11. The drug-releasable medical device according to claim 10, wherein a layer of the composition is formed on the surface.

12. The drug-releasable medical device according to claim 10, wherein the medical device contacts with a living body or is incorporated or indwelled in a living body.

13. The drug-releasable medical device according to claim 10, wherein the medical device is a stent, a catheter, a clip, an organ replacement medical device, a capsule sensor or an artificial organ.

14. A stent for treating coronary artery stenosis gradually releasing an argatroban (an antithrombin drug) or a sarpogrelate hydrochloride (an antiplatelet drug) or both of them from its surface.

15. The stent according to claim 14, wherein a release rate of both the argatroban and sarpogrelate hydrochloride is 1×10⁻³ μg/mm²-h to 1 μg/mm²-h on 21 days after indwelling the stent.

16. The stent according to claim 14, wherein the drug to be gradually released is carried in a polymeric material coated on a surface of a metal forming the stent.

17. The stent according to claim 16, wherein the polymeric material coated on the surface of the stent is amorphous.

18. The stent according to claim 16, wherein the polymeric material coated on the surface of the stent is an amorphous biodegradable polymeric material.

19. The stent according to claim 16, wherein the polymeric material further comprises a release auxiliary agent promoting the release of a drug to be carried.

20. The stent according to claim 14, wherein the surface of a metal forming the stent is a porous body and the drug to be gradually released is carried in the porous body.
